(19) United States
(12) Patent Application Publication
(10) Pub. No.: US 2010/0108061 A1
(43) Pub. Date: May 6, 2010

(54) EVAPORATOR DEVICE TO ASSIST BREATHING

(76) Inventor: Barry Paul Henderson, Derry (GB)

Correspondence Address: MONAHAN & MOSES, LLC, 13-B W. WASHINGTON ST., GREENVILLE, SC, 29601, US

(21) Appl. No.: 12/451,235

(22) PCT Filed: May 1, 2008
(86) PCT No.: PCT/GB2008/001530

§ 371 Date: Dec. 17, 2009

(30) Foreign Application Priority Data
May 1, 2007 (GB) 0708392.6

(51) Int. Cl. A61M 16/16

(52) U.S. Cl. 128/203.16; 128/203.26

(57) ABSTRACT

The present invention relates to an evaporator device to assist breathing comprising a low profile body adapted to house heating means (9), a liquid reservoir (8), an evaporation site and means (15) to supply liquid from the reservoir to the evaporation site such that the device can humidify the air in the immediate environment. The heating means may comprise PCMs, means to connect to external power, a heating mat and/or encapsulated materials which generate heat through an exothermic chemical reaction. The liquid may be water or a solution including or consisting of an active agent, for example a decongestant. The device may be used to produce nitric oxide to assist breathing.

The present invention relates to an evaporator device which is used to provide a conditioned microclimate of inhaled air. More particularly the invention relates to a personal evaporator device for use during periods of rest and sleep to improve breathing for an individual.

Nasal breathing by healthy individuals ensures that the lungs are well conditioned and therefore function well.¹ The nose performs a number of functions during breathing including warming, humidifying and releasing nitric oxide into inhaled air. Individuals who have difficulty breathing through their nose will not benefit from this functionality. Consequently mouth breathers have more poorly conditioned lungs.

A number of conditions including rhinitis and sinusitis can lead to the blocking of nasal airway passages. This leads to mouth breathing rather than nose breathing. Nose breathing is preferable as the nose conditions inhaled air so that it is warmer, filtered, more humid and enriched with nitric oxide thereby helping maintain good lung function.² Most asthmatics will have a tendency to mouth breathe often unconsciously reverting to this method at night or when suffering mild asthma. Mouth breathing is less desirable as air is not conditioned as it is when inhaled through the nose. For mouth breathers the inhaled air is less humid and exhaled air is more humid. This process leads to drying of the oral mucosa, the upper respiratory tract and the lungs that can cause cell damage and/or death in these areas. This damage can lead to an inflammation response that may trigger an asthmatic episode e.g. airway hyper-responsiveness (AHR). It also affects the ability to condition inhaled air and leaves the mucosa open to viral or bacterial infection.³

During sleep as there may be more drying whilst an individual is unconscious. During sleep mouth breathers lose moisture as described above which leads to discomfort, broken sleep, inflammation in the upper respiratory tract and asthmatic episodes. In addition to acute and chronic health problems associated with mouth breathing at night individuals may suffer discomfort resulting in broken sleep and tiredness.⁴

Such drying of the oral mucosa, upper respiratory tract and lungs may lead to one or more of the following conditions: asthma, dry mouth, oral infection, upper respiratory tract infection, bronchial infection, sleeplessness, vocal cord damage, hoarseness.

Nitric oxide is a well-known vasodilator as it has the ability to relax types of smooth muscle, including airway smooth muscle. Within the respiratory system nitric oxide acts as a bronchodilator and a broncho-protectant. It has bacteriostatic properties, that is, it inhibits the growth of bacteria and viruses and triggers the killing of bacteria and viruses by the white blood cells.⁵ As nitric oxide is vital in dilating the airways, it has been established that people with respiratory complaints could benefit from additional inhaled nitric oxide^{6,7}.

The present invention aims to provide a new ex-vivo evaporator device to replace and/or supplement nasal breathing. The device aims to help and prevent acute and chronic medical conditions associated with inhalation through the mouth (mouth breathing), rather than inhalation through the nose, for extended periods of time e.g. patients with sinusitis, rhinitis, asthma, or for short periods of time e.g. hyperventilation. The device also aims to treat those who are unable to effectively condition inhaled air due to damage or dysfunction in the nose. Asthmatics are known to be susceptible to exposure to cold, dry air.⁸ The device aims to alleviate discomfort felt by patients eg drying of the mouth and airways.

Currently the main therapy for asthmatics is the use of inhaled therapies including both broncho-dilators and preventatives (cortico-steroids). Although such therapeutics are very effective, there are a number of disadvantages. The primary disadvantage is that asthmatics continue mouth breathing rather than nasal breathing. Prolonged nasal breathing ensures that the lungs remain healthier and less prone to irritation by allergens. This is due to the fact that the air is warmed, humidified and enriched with Nitric Oxide at a level of approximately 5-50 parts per billion (ppb).^{9,10,11}

A particular aim of this invention is to provide a device that recreates nasal function and can be breathed over by an asthmatic thereby replacing lost nasal function or supplementing sub-optimal nasal function.

According to the present invention there is provided an evaporator device comprising a body adapted to house heating means, a liquid reservoir, an evaporation site and means to supply liquid from the reservoir to the evaporation site.

The body will preferably have a low profile shape to sit on a bed or pillow surface.

In one embodiment the heating means comprises an encapsulated phase change material (PCM). A Phase Change Material (PCM) is a substance with a high heat of fusion which, melting and solidifying at certain temperatures, is capable of storing or releasing large amounts of energy. The PCM employed may be either an organic compound based PCM or a salt-based PCM. The melting temperature of the PCM employed can be varied to achieve the rate of evaporation desired for example, 42° C., 58° C. or 80° C. These temperatures are not limiting and other temperatures can be employed depending on the PCM.

In an alternative embodiment the heating means comprises power charging means or means to connect to external power.

In a further alternative the heating means comprises a heating mat or encapsulated materials which generate heat through an exothermic chemical reaction.

Preferably the heating means is removeably locatable in the body.

Preferably the means to supply liquid from the reservoir is a wicking material.

In one embodiment the evaporation surface comprises a wicking material.

Preferably the reservoir is releasably engageable with the body.

The device works by producing a moist or warm and moist microenvironment which when breathed across will increase the levels of moisture or moisture and temperature of inhaled air. By raising humidity and/or temperature inhaled air is effectively conditioned.

The device may comprise an external power source.

The device may be used in isolation or be embedded in, attached to, or rested against a pillow, cushion, headrest, sofa, mattress or similar, or used with a portable handheld system.

In one embodiment the housing comprises a base unit capable of delivering heat to the evaporation site on a surface, an integral container holds the liquid reservoir; a wick (e.g. cotton, paper towel) is the means to enable transfer of this liquid from the reservoir to the evaporation surface. An optional power unit and/or controllable heating system can also be included.

The liquid may consist of water or a solution including or consisting of an active agent e.g. a decongestant.

The wick may also have integrated material or substances e.g. nitric oxide releasing hydrogel that will enable release active therapeutic agents e.g. physiological levels of nitric oxide at the evaporative surface. Nitric oxide is a broncho-protectant acting as both an anti-microbial and broncho-dilator.¹² Individuals will use the device to improve breathing through their nose or reduce the broncho-constriction and/or the potential for respiratory tract infections when breathing through their mouth.

The device may be integral to, or attached to, a pillow, cushion, headrest, sofa, mattress or similar, or wrap or cover for the above, or a portable handheld system.

The device will condition the lungs and reduce damage and dysfunction which would otherwise have occurred to the mucosal epithelium in the nose, mouth, upper respiratory tract and the lungs by raising the warming and humidifying inhaled air. The device will also enable the delivery of decongestants eg. Vick VapoRub (Levomenthol (2.75% w/w), camphor (5.00% w/w), eucalyptus oil (1.50% w/w) and turpentine oil (5.00% w/w) as the active ingredients or Olbas Oil (Cajuput Oil BPC 18.50%, Clove Oil BP 0.10%, Eucalyptus Oil BP 35.45%, Juniper Berry Oil BPC '49 2.70%, Menthol BP 4.10%, Dementholised Mint Oil BP 35.45%, Wintergreen Oil BPC '49 3.70%) or physiological levels of nitric oxide to the lungs for a prolonged time period thereby improving lung condition and function.

Typically the device will consist of a base unit capable of delivering heat to an evaporative surface. The base unit will ideally be a polymer insulated aluminium heat sink containing phase change material. The polymer can be polypropylene or any other suitable polymer.

The aluminium heat sink can employ an internal honeycomb structure to ensure that heat energy is efficiently transferred from a cartridge heater to the PCM and from the PCM to the evaporative surface.

Alternatively the heat energy may be dissipated through the PCM by means of metallic fins or constructs, the nature, shape and quantity of which provide optimal and efficient heat distribution whilst minimising the thermal mass of the conducting material.

This base unit will be capable of being quickly heated or charged for a short time period e.g. 15-60 minutes using an appropriate cartridge heater. The base unit will be designed in order that heat will be released slowly over an extended period depending upon the volume and melting temperature of the phase change material employed e.g. 30 minutes-5 hours, allowing prolonged evaporation of any liquid and creating a humid microenvironment.

An integral container holds the reservoir of liquid. The liquid will consist of water or water and an active agent.

The wick of for example cotton or paper towel enables transfer of this liquid from the container to the evaporation surface.

The device can include an optional power unit and controllable heating system.

A wick most likely consisting of paper or cotton will draw water or water including an active agent from a reservoir integrated or attached to the base unit to a heated evaporative surface. The wick may also have integrated material or substances e.g. nitric oxide releasing hydrogel that will enable release active therapeutic agents e.g. physiological levels of nitric oxide at the evaporative surface.¹³ Using the device the end user will experience improved breathing through the nose or reduction in broncho-constriction whilst mouth breathing.

As the device will have a low profile enabling it to be embedded in or used in conjunction with a pillow, a headrest, or similar it may be used while resting or sleeping in order to raise the humidity of inhaled air. The device will raise humidity and/or temperature of the air at the active site. This surface will be positioned flush or almost flush against the base. Conditioned air will be inhaled by breathing across the active site either through the nose and/or mouth. The device will be capable of producing warm, humidified, nitric oxide enriched micro-environment at the evaporative surface. The device will raise humidity and/or temperature and/or nitric oxide levels and/or decongestants contained in the air at the evaporative surface.

An optional power unit and controllable heating system may be included to control the temperature of the liquid repository and therefore humidity levels at the evaporation site.

The device can allow the delivery of a therapeutic agent through the use of nitric oxide releasing biomaterials or nitric oxide releasing natural materials to prevent against airway hyper-responsiveness (AHR). Release of such agents will be activated by increased moisture, heat or light levels. This may be achieved by a variety of methods including the use of water insoluble polymeric NONOate complexes contained in an absorbent material (the wick) which will release nitric oxide upon diffusion with water¹⁴, polymerizable biodegradable hydrogels, polymeric materials etc^{16,17,18,19,20,21}.

The wick and/or the biomaterial will release nitric oxide at levels that mimic physiological conditions in the lungs i.e. 5-50 ppb. Specialist wicks and/or the biomaterials may also be developed that release much greater levels of nitric oxide in short time periods to mimic the release of nitric oxide in the para-nasal sinuses, at levels between 4000 and 7000 ppb¹⁵ which would be used as a non-pharmaceutical method to clear nasal constriction.

Preferably the biomaterial will release between 200 and 8000 ppb nitric acid to allow an effective amount to be delivered to the lungs taking into account the amount that will be dissipated before it reaches the lungs.

Timed release of bursts of nitric oxide from the device may be used to naturally mimic nasal breathing.

Suitable biomaterials that release nitric oxide maybe similar to or based on those developed by Rice University and Michigan University for in vivo use as vascular implants.

This device will be capable of being adapted for use as a handheld system to humidify a microenvironment which one could breathe across.

The device is further described with reference to the following non limiting figures.

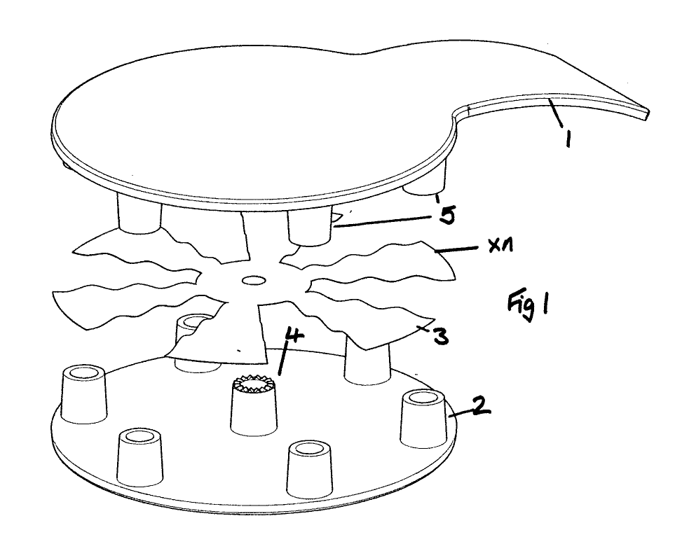

FIG. 1 illustrates the top and bottom of the housing for the PCM material and the foil used to dissipate heat.

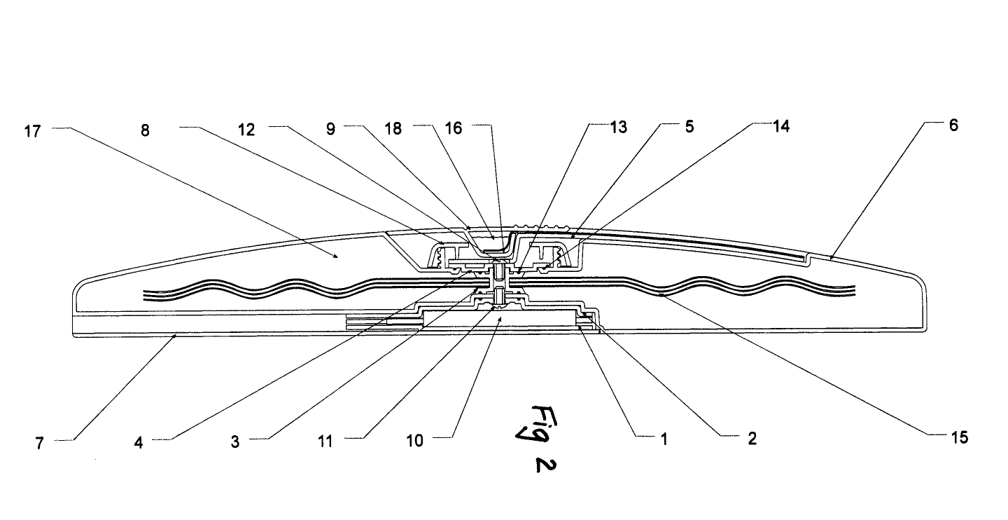

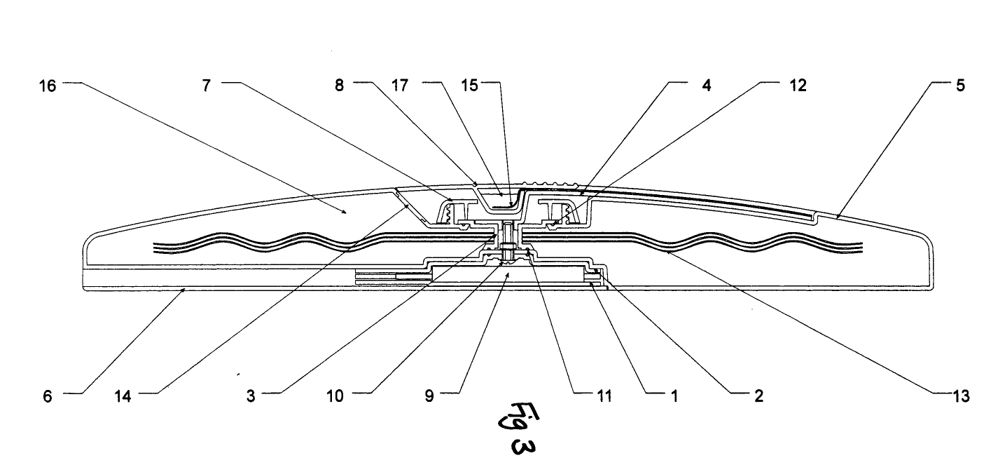

FIGS. 2 and 3 are drawings of the structural details of two similar embodiments of the breathing device.

The figures illustrate non limiting embodiments of the evaporator of the invention which is adapted to condition air being inhaled by an individual, particularly for use during periods of rest or sleep. The device is intended to be low profile in shape and can be positioned in close proximity to the individual, such as on a bed or bedside.

The device comprises a body which is preferably moulded from a polymer with an internal metal casing. The body defines the reservoir, an evaporation site and a heating chamber and includes means to supply liquid in the reservoir to the evaporation site.

In use the reservoir contains a liquid, generally water or a solution of a medicament/active ingredient.

Whereas FIGS. 1 to 3 show similar embodiments of the device in detail, it will be appreciated that other embodiments will have similar features in different designs without departing from the main features of the invention. Embodiments can be designed to be portable and can be used with or without the incorporation of a face mask.

The heating means can provide heat by various means or holds materials such as a phase change material (PCM) which can be charged to provide heat. PCMs are, for example, paraffin wax based materials with phase transition temperatures in the range from 25 to 125 degrees centigrade. Such materials are excellent for storing large amounts of thermal energy in a relatively small volume. Charging the PCM can be achieved by electrical means, by immersion in heated liquid or via a heated mat. The charging method may be integral or separate to the device and may require insulation or isolation from the rest of the device for safety reasons and to prevent malfunction.

The device further comprises supply means typically in the form of a wick which extends from the reservoir to the evaporation site. The wick can be of any suitable material, for example cotton or paper towel, which will allow liquid to be transferred from the reservoir to the evaporation site. The wick is preferably formed from a fabric with good capillary attraction to provide an adequate rate of transfer of liquid. Other suitable supply means may be used in place of the wick.

The PCM can be present in an arrangement as shown in FIG. 1 wherein the chamber, preferably of aluminium casing has a top part 1 and lower part 2 which encloses the PCM and conductive foil 4 which extends from in or around the central axle 3 into the PCM material and the foil and casing conduct heat to charge the PCM. There may be several layers of aluminium foil strands extending into the PCM material in the chamber. The chamber can be designed with channels 5 running into or through the chamber to conduct heat and provide stability to the structure.

The PCM chamber may alternatively be of a design not illustrated which has an internal structure comprising of hexagonal channels which assist in the effective transfer of heat to and from the PCM material. These structures may also have cut out channels to assist PCM flow (in liquid state) during manufacture. These channels prevent air pockets and non uniform PCM filling. The chamber space, shape and the array can be arranged to ensure heat transfer is focused and channeled effectively. The use of hexagonal channels allows for optimum heat transfer whilst maximising the volume of PCM housed within the structure. This ensures a shallow design can be employed thereby improving usability by a sleeping or resting person.

In use the heater heats the aluminium casing and the foils which in turn heat the PCM. The metal casing and foils accelerate the heating process and make it more consistent across the entire PCM. The wicking surface (also metal is heated). The wick material which sits in contact with this surface is also dipped into the water. A wicking process occurs and the liquid is drawn down the wick and then in contact with the wicking surface. The wicking surface's heat turns this water into steam.

FIGS. 2 and 3 represent two similar versions of the device as proposed for manufacture. The parts of the devices are set out as follows.

### FIG. 2

1 Heater cover base.
   Aluminum enclosure. Heater lies with flat surface in contact with this part.
2 Heater Cover top
   Aluminum enclosure
3 Centre fixing axle
   Aluminum lathed profile which clamps together part 5 between parts 2 and 3. PCM is sealed in place using parts 11 (×2)
4 Centre axle top cap
5 Wicking surface
   Aluminum part which slots into a cylindrical hole in part 3. It seals against part 5 with part 12 under compression from part 7
6 PCM housing
   Acetal (or similar) heat resilient polymer either rotationally moulded to a 2 part injection moulding ultrasonically welded together to provide a sealed cavity for retention of PCM.
7 Heater/electronics trim plate
   Polymer (Acetal or similar) trim cover to protect from heater and to channel electronic wiring.
8 Top fixing dial
   Acetal threaded dial clamp. Presses down on part 3 and compresses part 5 upwards sealing with part 12.
9 H20 reservoir
   PC/ABS transparent water reservoir. Flip lid for easy refill, even when in use. Has a wicking cover to keep wick in place and offers some protection from hot surface. Features a live hinge. 2 part ultrasonic welded together.
10 Heater

### Off the Shelf

11 M10 fixing bolt
   Off the shelf. Approx 12-15 mm (may need to reduce to 8 mm. Larger diameter preferred for greater compression force under sealing
12 M10 fixing bolt
   Off the shelf. Approx 12-15 mm (may need to reduce to 8 mm. Larger diameter preferred for greater compression force under sealing
13 M20 ‘O’ Ring
   Off the shelf—Major Diameter, cross sectional diameter and detail sealing cavity to be sourced when final size has been specified.
14 M40 ‘O’ Ring
   Off the shelf—Major Diameter, cross sectional diameter and detail sealing cavity to be sourced when final size has been specified.
15 Conductive foil
   0.1 mm foils compressed between parts 3 and 5. However they are not compressed between the ‘O’ Ring seal. A shoulder is required to keep their location separate.
16 Wicking material

### FIG. 3

1 Heater cover base.
   Aluminum enclosure. Heater lies with flat surface in contact with this part.
2 Heater Cover top
   Aluminum enclosure
3 Centre fixing axle
   Aluminum lathed profile which clamps together part 5 between parts 2 and 3. PCM is sealed in place using parts 11 (×2)
4 Wicking surface
   Aluminum part which slots into a cylindrical hole in part 3. It seals against part 5 with part 12 under compression from part 7
5 PCM housing
   Acetal (or similar) heat resilient polymer either rotationally moulded to a 2 part injection moulding ultrasonically welded together to provide a sealed cavity for retention of PCM.
6 Heater/electronics trim plate
   Polymer (Acetal or similar) trim cover to protect from heater and to channel electronic wiring.
7 Top fixing dial
   Acetal threaded dial clamp. Presses down on part 3 and compresses part 5 upwards sealing with part 12.
8 H20 reservoir
   PC/ABS transparent water reservoir. Flip lid for easy refill, even when in use. Has a wicking cover to keep wick in place and offers some protection from hot surface. Features a live hinge. 2 part ultrasonic welded together.
9 Heater

### Off the Shelf

10 M10 fixing bolt
   Off the shelf. Approx 12-15 mm (may need to reduce to 8 mm. Larger diameter preferred for greater compression force under sealing
11 M20 ‘O’ Ring
   Off the shelf—Major Diameter, cross sectional diameter and detail sealing cavity to be sourced when final size has been specified.
12 M40 ‘O’ Ring
   Off the shelf—Major Diameter, cross sectional diameter and detail sealing cavity to be sourced when final size has been specified.
13 Conductive foil
   0.1 mm foils compressed between parts 3 and 5. However they are not compressed between the ‘O’ Ring seal. A shoulder is required to keep their location separate.
14 PCM access point
   Polymer seal cap—friction welded after PCM has been included.
15 Wicking material

The heat array may be enclosed in aesthetic and ergonomic polymer housing all around the heat array creating an insulation pocket, preventing heat loss. It may be open at top (around the evaporative surface) allowing greater control of the heat transfer to this location. These covers are shaped to be non-intrusive and capable of lying on the bed in any arrangement (similar to a mouse mat on a table). They provide a gentle slope so that the nose and mouth areas can come closer to the evaporative surface comfortably. The base of the unit can have a non slip texture.

The seals should be designed to be splash resistant but not completely seal the unit as this would affect the wicking process. A ribbed structured inside the aperture can clasp the wick without affecting the flow properties.

For the embodiments shown, regardless of end use, such as for a bed edge concept and for a bed top concept, similar assembly concepts can be used.

Those who would benefit from using such a device would include individuals with a tendency to mouth breath such as those with asthma, rhinitis, sinusitis. The device could provide non pharmaceutical therapy of for asthmatics.

Also, the device would be useful for other conditions where dehydration of the drying of oral mucosa and/or upper respiratory tract and/or lungs exacerbates a medical condition eg. cystic fibrosis and for patients undergoing therapy that may result in the drying of oral mucosa and/or upper respiratory tract and/or lungs eg. cancer patients.

Infants and pediatric patients are more likely to suffer from and require treatment for dehydration and the device would be of use in assisting them. Also, individuals subjected to dry and dusty, dry and hot, dry and cold, dry environments, for example airline passengers or employees, those in mining industry, nanofabrication workers (microchip industry) etc could benefit their health by using the device. Similarly, those working with harmful solvents e.g. furniture workers, petrochemical industry and individuals who wish to protect themselves from or treat themselves from dehydration of their vocal cords eg. singers, vocalists, call centre operators could benefit from using the device.

The present invention is not limited to the embodiments described herein which may be amended or modified without departing from the scope of the present invention.

### REFERENCES

¹ Human Anatomy and Physiology, Elaine N. Marieb, 6^{th} Edition.
² Measurement of nitric oxide in human nasal airway. M. Imada, J Iwamoto, S Nonaka, Y Kobayashi, and T Unno
³ Increased net water loss by oral compared to nasal expiration in healthy subjects. Svensson S, Olin A C, Hellgren J. Rhinology 2006; 44: 74-7
⁴ The effects of partial and complete mechanical occlusion of the nasal passages on sleep structure and breathing in sleep. Lavie P, Fischel N, Zomer J, Eliaschar I. Acta Otolaryngologica 1983; 95: 161-166
⁵ Effects of nitric oxide and nitrogen dioxide on bacterial growth. Applied and Environmental Microbiology. Mancinelli R, McKay C. 1983; 46: 198-202
⁶ Nitric oxide as a therapeutic agent. 1994 May; 17(5):227-8.
⁷ Inhaled nitric oxide for the treatment of ARDS Lohbrunner H, Deja M, Busch T, Spies C D, Rossaint R, Kaisers U. 2004 August; 53(8):771-82; quiz 783-4.
⁸ Airway cooling in asthmatic and non-asthmatic subjects during nasal and oral breathing. Griffin M P, McFadden E R, Ingram R H Journal of Allergy and Clinical Immunology 1982; 69: 354-9
⁹ http://www.parkhurstexchange.com/archives/update/2006/ud_{—}02_vol14.html
¹⁰ http://www.medscape.com/viewarticle/466828_{—}3
¹¹ http://cat.inistfr/?aModele=afficheN&cpsidt=1965895
¹⁵ The Paranasal Sinuses as Reservoirs for Nitric Oxide Authors: Andersson J. A.1; Cervin A.2; Lindberg S.2; Uddman R.1; Olaf Cardell L.1 Source: Acta Oto-Laryngologica, Volume 122, Number 8, 1 Dec. 2002, pp. 861-865(5) Publisher. Taylor and Francis Ltd
¹⁶ Nitric Oxide-Producing Hydrogel Materials U.S. Pat. No. 7,052,711 Inventors: West; Jennifer L. (Pearland, Tex.), Masters; Kristyn Simcha (Northglenn, Colo.)
¹⁷ Nitric Oxide Releasing Polymers Incorporating Diazeniumdiolated Silane Derivatives U.S. Pat. No. 6,841,166 Inventors: Zhang; Huiping (Mount Vernon, Ind.), Meyerhoff; Mark E. (Ann Arbor, Mich.)
¹⁸ Two Dimensional Polymer That Generates Nitric Oxide U.S. Pat. No. 6,951,902 Inventors: McDonald; William F. (Utica, Ohio), Koren; Amy B. (Lansing, Mich.)
¹⁹ Polymeric Material That Releases Nitric Oxide U.S. Pat. No. 5,994,444 Inventors: Trescony; Paul (Champlin, Minn.), Rohly; Ken (Lino Lakes, Minn.), Dror; Michael (Parker, Co) Abstract
²⁰Nitric Oxide Releasing Polymers to Treat Restinosis and Related Disorders U.S. Pat. No. 5,650,447 Inventors: Keefer, Larry K. (Bethesda, Md.), Hutsell; Thomas C. (North Oaks, Minn.)
²¹Nitric Oxide Releasing Amidine Diazeniumdiolates, Compositions and uses thereof and method of making same U.S. Pat. No. 6,949,530 Inventors: Hrabie; Joseph A. (Frederick, Md.), Keefer; Larry K. (Bethesda, Md.), Arnold; Ernst V. (Hagerstown, Md.
¹² Inhalation of nasally derived nitric oxide modulates pulmonary function in humans. Lundberg J O, Settergren G, Gelinder S, Lundberg J M, Alving K, Weitzberg E Acta Physiologica Scandinavica 1996; 158: 343-7
¹³ Nitric oxide-producing hydrogel materials: U.S. Pat. No. 7,052,711. Inventors: West Jennifer L. (Pearland, Tex.), Masters; Kristyn Simcha (Northglenn, Colo.)
¹⁴ http://www.temarex.com/wound.htm

What I claim is:

1. An evaporator device to assist breathing comprising a body adapted to house heating means, a liquid reservoir, an evaporation site and means to supply liquid from the reservoir to the evaporation site such that the device is capable of being activated to humidify the air in the immediate environment.

2. A device as claimed in claim 1 wherein the heating means comprises an encapsulated phase change material (PCM), a substance with a high heat of fusion which, melting and solidifying at certain temperatures, is capable of storing or releasing energy.

3. A device as claimed in claim 1 wherein the heating means comprises power charging means, means to connect to external power, a heating mat and/or encapsulated materials which generate heat through an exothermic chemical reaction.

4. A device as claimed in claim 1 wherein the heating means is removeably locatable in the body.

5. A device as claimed in claim 1 wherein the means to supply liquid from the reservoir and/or the evaporation surface comprise a wicking material.

6. A device as claimed in claim 1 comprising a controllable heating system.

7. A device as claimed in claim 1 wherein the liquid consists of water or a solution including or consisting of an active agent, for example a decongestant.

8. A device as claimed in claim 1 wherein the wick contains an integrated material or substances that will enable release of active therapeutic agents at the evaporative surface.

9. A device as claimed in claim 1 wherein the device releases nitric oxide.

10. A device as claimed in claim 1 wherein the body is a low profile shape capable of sitting on a bed or pillow or integral to, or attachable to, a pillow, cushion, headrest, sofa, mattress or similar, or wrap or cover for the above, or a portable handheld system.

11. A device as claimed in claim 1 wherein the heating means is an aluminium heat sink has an internal honeycomb like structure.

12. A device as claimed in claim 1 wherein the heating means comprising metallic fins or constructs for heat dissipation.

13. A device as claimed in claim 1 comprising a controllable heating system to control the temperature of the liquid repository and therefore humidity levels at the evaporation site.

14. Use of nitric oxide releasing biomaterials or nitric oxide releasing natural materials in an ex vivo device to prevent against airway hyper-responsiveness (AHR) wherein release of such agents will be activated by increased moisture, heat and/or light levels.

15. Use as claimed in claim 14 wherein the materials are NONOate complexes.

16. Use as claimed in claim 14 wherein the materials are contained in an absorbent material which is able to release nitric oxide.
